(19) United States
(12) Patent Application Publication
(10) Pub. No.: US 2013/0035568 A1
(43) Pub. Date: Feb. 7, 2013

(54) INFORMATION PROCESSING APPARATUS, INFORMATION PROCESSING METHOD, PROGRAM, AND INFORMATION PROCESSING SYSTEM

(71) Applicants: Yoichi TORIUMI, Tokyo (JP); Tatsuya SUZUKI, Kanagawa (JP); Hirokazu IMAI, Chiba (JP); Hideo KAWABE, Saitama (JP); Kenichi KABASAWA, Saitama (JP); Masatoshi UENO, Kanagawa (JP)

(72) Inventors: Yoichi TORIUMI, Tokyo (JP); Tatsuya SUZUKI, Kanagawa (JP); Hirokazu IMAI, Chiba (JP); Hideo KAWABE, Saitama (JP); Kenichi KABASAWA, Saitama (JP); Masatoshi UENO, Kanagawa (JP)

(73) Assignee: Sony Corporation, Tokyo (JP)

(21) Appl. No.: 13/538,600

(22) Filed: Jun. 29, 2012

(30) Foreign Application Priority Data
Aug. 1, 2011 (JP) 2011-168191

(51) Int. Cl. A61B 5/1455; A61B 5/0295

(52) U.S. Cl. 600/322

(57) ABSTRACT

Provided is an information processing apparatus including a judgment unit for using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

### BACKGROUND

The present disclosure relates to an information processing apparatus, an information processing method, a program, and an information processing system.

In the related art, an apparatus is proposed which non-invasively measures the concentration of a specific component (a component that flows in a blood vessel, for example) in a living body by irradiating a body surface of a subject with light, and measuring reflected light from the body surface.

For example, in JP 2010-526646A, a method is disclosed in which the concentration of glucose that exists in the blood (that is, blood sugar level) of a subject is measured by using an optical sensor or a spectrometer.

### SUMMARY

By the way, a case is considered, for example, where the concentration of a specific component that flows in a blood vessel is measured based on reflected light from a body surface. In this case, the concentration of the specific component is calculated according to Beer-Lambert law wherein “an absolute amount of a specific component that exists in a light irradiation region=the concentration of a specific component in a blood vessel×the distance by which the irradiation light transmits through the blood vessel”. In this case, “an absolute amount of a specific component that exists in a light irradiation region” corresponds to a value calculated based on an amount of the reflected light, and the concentration of the specific component in the blood vessel is often calculated based on an assumption that “a distance by which the irradiation light transmits through the blood vessel” does not vary so much.

However, “an absolute amount of the specific component that exists in a light irradiation region” varies depending on a condition of a subject such as dilation/constriction of a blood vessel or increase/decrease of blood flow. Therefore, when the calculation is carried out without considering the condition of the blood vessel or the blood flow, the concentration of the specific component might be overestimated or underestimated. Therefore, if the condition of the subject such as the dilation/constriction of the blood vessel or the increase/decrease of the blood flow can be easily specified, it becomes possible to measure the concentration of the focused specific component more accurately.

Therefore, the present disclosure provides an information processing apparatus, an information processing method, a program, and an information processing system, which are capable of judging a condition of a blood vessel or blood flow, such as dilation/constriction of the blood vessel or increase/decrease of the blood flow.

According to an embodiment of the present disclosure, there is provided an information processing apparatus which includes a judgment unit for using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

Further, according to another embodiment of the present disclosure, there is provided an information processing method which includes using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

Still further, according to another embodiment of the present disclosure, there is provided a program which causes a computer to execute a function for using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

Still further, according to another embodiment of the present disclosure, there is provided an information processing system which includes a measurement unit for generating measurement data in relation to reflectance of light by irradiating a surface of a living body of a subject with light of a predetermined wavelength, and by detecting light reflected at the living body, and an information processing apparatus including a judgment unit for judging a condition of a blood vessel and/or a condition of blood flow of the living body in accordance with a color phase corresponding to the reflectance of light by using the measurement data in relation to the reflectance of light generated by the measurement unit, the measurement unit including a light-receiving element at which light from a measurement target region is imaged, the surface of the living body being placed on the measurement target region, and a plurality of light-emitting elements arranged on a periphery of the light-receiving element in a circular manner for irradiating the measurement target region with light of a predetermined wavelength, the plurality of light-emitting elements being arranged to incline with respect to a normal line of the measurement target region so that a central line of irradiated light from each of the light-emitting elements passes through an approximate center of the measurement target region.

According to the embodiments of the present disclosure, the color phase corresponding to the reflectance of light is specified based on the measurement data in relation to the reflectance of light obtained by irradiating the surface of the living body of the subject with the light of the predetermined wavelength, and the conditions of the blood vessel or the blood flow of the living body is judged in accordance with the specified color phase.

According to the present disclosure as described above, the conditions of the blood vessel or the blood flow such as the dilation/constriction of the blood vessel and the increase/decrease of the blood flow can be judged.

### BRIEF DESCRIPTION OF THE DRAWINGS

FIG. 1 is a graphical representation illustrating findings of the concentration of a blood component specified based on reflectance;

FIG. 2 is an explanatory diagram showing an information processing system according to a first embodiment of the present disclosure;

FIG. 3 is a block diagram showing a configuration of an information processing apparatus according to the embodiment;

FIG. 4 is a graphical representation showing a wavelength property of the reflectance of the skin;

FIG. 5 is a graphical representation showing the wavelength property of the reflectance of the skin;

FIG. 6 is an explanatory diagram illustrating a judgment process implemented in the information processing apparatus according to the embodiment;

FIG. 7 is an explanatory diagram illustrating the judgment process implemented in the information processing apparatus according to the embodiment;

FIG. 8 is an explanatory diagram illustrating the judgment process implemented in the information processing apparatus according to the embodiment;

FIG. 9A is an explanatory diagram schematically showing an example of a measurement unit according to the embodiment;

FIG. 9B is an explanatory diagram schematically showing an example of the measurement unit according to the embodiment;

FIG. 10 is an explanatory diagram schematically showing an example of the measurement unit according to the embodiment;

FIG. 11 is an explanatory diagram schematically showing an example of a measurement unit according to the embodiment;

FIG. 12 is an explanatory diagram schematically showing an example of the measurement unit according to the embodiment;

FIG. 13 is a flow diagram showing an exemplary flow of an information processing method according to the embodiment; and

FIG. 14 is a block diagram showing a hardware configuration of the information processing apparatus according to the embodiment of the present disclosure.

### DETAILED DESCRIPTION OF THE EMBODIMENTS

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

The description will be given in the following order.

### (1) Findings of Concentration of Blood Component Specified Based on Reflectance

### (2) First Embodiment

### (2-1) Information Processing System

### (2-2) Information Processing Apparatus

### (2-3) Example of Measurement Unit

### (2-4) Information Processing Method

### (3) Hardware Configuration of Information Processing Apparatus According to Embodiment of Present Disclosure

### (Findings of Concentration of Blood Component Specified Based on Reflectance)

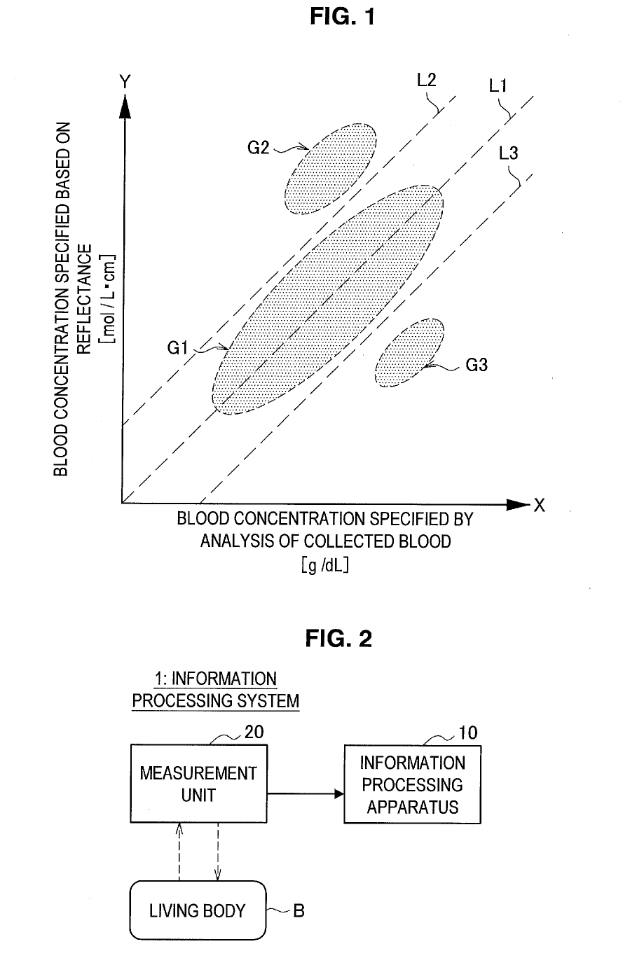

Prior to describing an information processing system, an information processing apparatus, and an information processing method according to an embodiment of the present disclosure, findings in relation to the concentration of a blood component specified based on reflectance obtained by the inventors will be briefly described with reference to FIG. 1. FIG. 1 is a graphical representation illustrating findings of the concentration of a blood component specified based on reflectance.

The inventors carried out the following study in order to verify measurement accuracy of a technology for measuring the concentration of a blood component (in the following example, the concentration of hemoglobin in the blood) based on reflected light from the skin by irradiating the skin of a subject with light of within a visible light band.

That is, with respect to the statistically sufficient number of subjects, reflectance is measured by irradiating a portion of a body surface with the light of within the visible light band, and by collecting blood immediately after the measurement, the concentration of hemoglobin calculated based on the reflectance and the concentration of hemoglobin obtained by analyzing the collected blood are respectively specified.

A scatter diagram which indicates a correlation of the concentrations specified by the two kinds of methods can be obtained by axes of coordinate in which the concentration specified by the analysis of the collected blood is shown on the horizontal axis and the concentration specified based on the reflectance is shown on the vertical axis, and points corresponding to the specified two kinds of concentrations are plotted thereon.

FIG. 1 is a schematic diagram showing the scatter diagram produced based on data of the actually obtained concentrations. In FIG. 1, a line L1 is a straight line represented by Y=X, and a line L2 and a line L3 are straight lines respectively representing allowable limits of error. When magnitudes of the two kinds of actually obtained concentrations were plotted, as schematically shown in FIG. 1, it became clear that the points plotted on the scatter diagram were classified into any one of groups G1 to G3.

In FIG. 1, the plot that belongs to the group G1 corresponds to a plot for which the concentration specified by the blood collection and the concentration calculated from the reflectance were nearly equal, and a difference between them fell within the allowable limit of error. Most of the obtained data turned out to belong to the group G1. However, among the obtained data, there were some data that belonged to the groups G2 and G3. Among such data, the plot that belongs to the group G2 corresponds to a case in which the concentration calculated from the reflectance was larger than the concentration specified by the blood collection, in other words, the concentration was overestimated by the calculation method based on the reflectance. Also, the plot that belongs to the group G3 corresponds to a case in which the concentration calculated from the reflectance was smaller than the concentration specified by the blood collection, in other words, the concentration was underestimated by the calculation method based on the reflectance.

Here, when the inventors carried out a study regarding the subjects, whose results turned out to belong to the groups G2 and G3, it was found out that the subject who belonged to the group G2 was a person who was diagnosed to have high blood pressure at a blood pressure test, and the subject who belonged to the group G3 was a person who was diagnosed to have low blood pressure at a blood pressure test. The blood pressure tests were separately carried out from the measurement of the reflectance. The person having high blood pressure has a tendency in which a blood vessel is dilated and blood flow is increased; whereas the person having low blood pressure has a tendency in which the blood vessel is constricted and the blood flow is decreased.

When the inventors focused on spectrums of the reflectance corresponding to the data which belonged to the group G2 or G3 based on the findings, they found out that these two groups had a tendency of distinctive reflectance, as described later. Therefore, as a result of a further study based on the obtained findings, the inventors have found out a method described later for judging dilation/constriction of the blood vessel or increase/decrease of the blood flow by using the reflectance obtained by the measurement.

### First Embodiment

### <Information Processing System>

First of all, hereinafter, an information processing system according to a first embodiment of the present disclosure will be described with reference to FIG. 2. FIG. 2 is an explanatory diagram showing the information processing system according to the embodiment.

An information processing system 1 according to the present embodiment includes an information processing apparatus 10 and a measurement unit 20 as shown in FIG. 2.

The information processing apparatus 10 judges a condition of blood flow or a blood vessel positioned in the vicinity of a body surface (the skin) of a living body B by using measurement data in relation to reflectance of the body surface of the living body B, the measurement data being measured by the measurement unit 20 described below. Further, the information processing apparatus 10 is also capable of calculating the concentration of a specific component which exists inside the living body by using the measurement data in relation to the reflectance measured by the measurement unit 20. In this case, the information processing apparatus 10 corrects the calculated concentration of the specific component in accordance with a judgment result in relation to the condition of the blood vessel or the blood flow.

Further, the information processing apparatus 10 is capable of providing various services to the user of the information processing apparatus 10 by executing various applications. In executing the application, the information processing apparatus 10 is capable of using the judgment result in relation to the condition of the blood vessel or the blood flow as an execution parameter of the execution of the application.

A detailed configuration of the information processing apparatus 10 will be described later again.

The measurement unit 20 measures the reflectance of light at the body surface (skin) by irradiating the body surface of the living body B with light fallen within a predetermined wavelength band, and by detecting reflected light from the body surface. A known device can be used as the measurement unit 20 as long as it is capable of measuring the reflectance of light at the body surface of the living body. Examples of the measurement unit may include a spectrometer which measures the magnitude of reflectance for each wavelength by irradiating the body surface with white light, for example, and by dispersing reflected light from the body surface. Further, as the measurement unit 20, as described below by a specific example, a measurement device can be used in which the body surface is selectively irradiated with a proper amount of light of a predetermined wavelength specialized for a property of the skin so as to lower the degree of invasion.

After generating measurement data in relation to the reflectance of irradiated light at the skin, the measurement unit 20 outputs the generated data to the information processing apparatus 10.

Note that, in the example shown in FIG. 2, the information processing apparatus 10 and the measurement unit 20 are described as being independent of each other. However, the function of the information processing apparatus 10 according to the present embodiment may be realized as a function of a control unit for controlling operation of the measurement unit 20, or may be implemented in any computer provided within a case of the measurement unit 20.

### <Information Processing Apparatus>

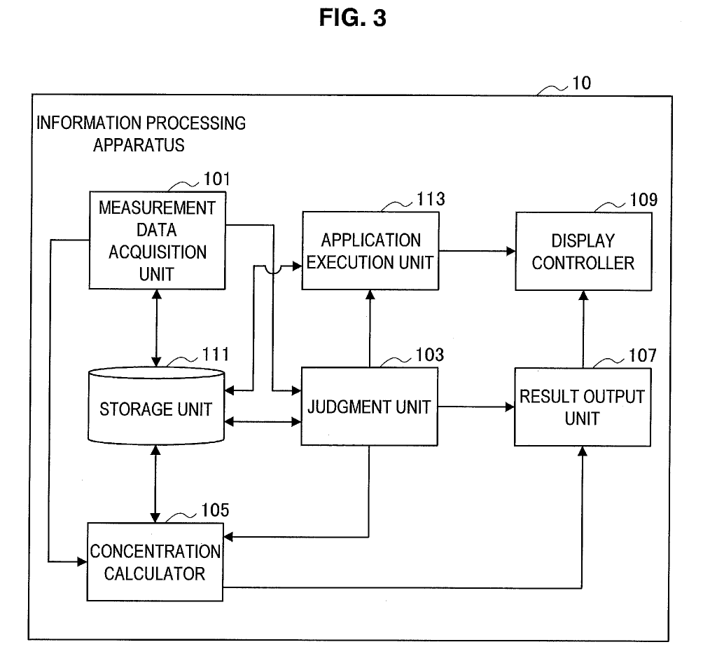

Next, the information processing apparatus according to the present embodiment will be described in detail with reference to FIGS. 3 to 8.

FIG. 3 is a block diagram showing a configuration of the information processing apparatus 10 according to the embodiment. As shown in FIG. 3, the information processing apparatus 10 includes a measurement data acquisition unit 101, a judgment unit 103, a concentration calculator 105, a result output unit 107, a display controller 109, and a storage unit 111. Further, the information processing apparatus 10 may includes an application execution unit 113 in addition to the above processing units.

The measurement data acquisition unit 101 is realized by, for example, a CPU (Central Processing Unit), a ROM (Read Only Memory), a RAM (Random Access Memory), and a communication unit. The measurement data acquisition unit 101 acquires measurement data from the measurement unit 20 in relation to the reflectance of light at the skin measured by the measurement unit 20.

Upon acquiring the measurement data in relation to the reflectance from the measurement unit 20, the measurement data acquisition unit 101 outputs the acquired data to the judgment unit 103 and the concentration calculator 105 described below. Also, the measurement data acquisition unit 101 may store the acquired measurement data as history information in the storage unit 111 in association with time information regarding time and date when the data was acquired.

The judgment unit 103 is realized by, for example, a CPU, a ROM, and a RAM. The judgment unit 103 judges a condition of a blood vessel or blood flow of the living body in accordance with a color phase corresponding to the reflectance of light by using the measurement data in relation to the reflectance of light at the skin, the measurement data being output from the measurement data acquisition unit 101.

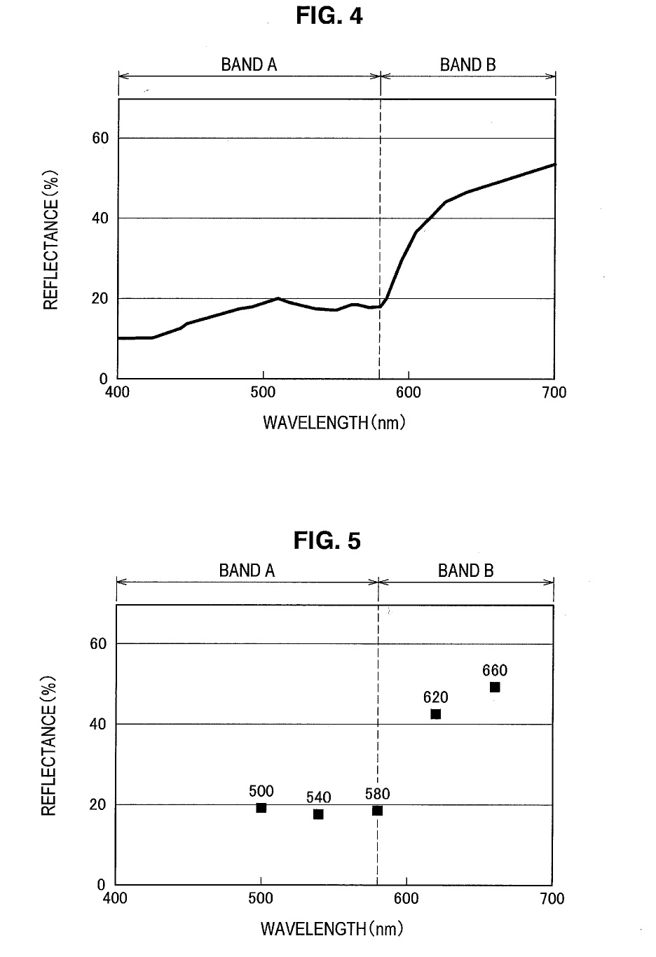

Hereinafter, a judgment process carried out in the judgment unit 103 according to the present embodiment will be described in detail with reference to FIGS. 4 to 8.

FIG. 4 is a graphical representation showing a measurement result of the reflectance of the human skin that was measured within a visible light band (400 to 700 nm). As is clear from FIG. 4, the reflectance of the human skin gently increases within the band in the vicinity of 400 to 500 nm. Then, until about the wavelength of 580 nm, the reflectance slightly decreases, and in the vicinity of wavelength of 580 to 650 nm, the reflectance sharply increases. The human skin exhibits a distinctive reflectance property as shown in FIG. 4. Here, the wavelength band B between over the wavelength of 580 to in the vicinity of 650 nm where the reflectance sharply increases corresponds to a color phase of red.

Focusing on the distinctive reflectance property of the human skin shown in FIG. 4, the judgment unit 103 of the present embodiment uses the reflectance of the band B corresponding to red (for example, a band over the wavelength 580 nm) and a band A positioned at a shorter wavelength side than the band B (for example, a band of the wavelength 580 nm or less) to judge dilation/constriction of the blood vessel and/or increase/decrease of the blood flow.

Here, the judgment unit 103 according to the present embodiment may use, as described in FIG. 4, measurement data obtained by measuring continuous transition of the reflectance for each wavelength (in other words, reflectance spectrum of the human skin), or may use a reflectance value of a specific wavelength instead of the continuous measurement data (in other words, measurement data that was measured in a discrete manner). It can be properly set which reflectance of which wavelength is focused when the discretely measured measurement data is used; however, it is preferable to use the reflectance at distinctive wavelength positions among the spectrums shown in FIG. 4.

Positions of the distinctive wavelengths among the spectrums shown in FIG. 4 are a position of 580 nm from which the reflectance sharply rises, a position between 400 and 500 nm where the reflectance gently increases, and a position where the reflectance sharply increases. Examples of the positions of the distinctive wavelengths may include five positions (500 nm, 540 nm, 580 nm, 620 nm, and 660 nm), as shown in FIG. 5. Here, the wavelength of 500 nm corresponds blue, the wavelength of 540 nm corresponds green, and the wavelength of 580 nm corresponds yellow. Also, the wavelengths of 620 nm and 660 nm correspond to red. Among these wavelengths, the three wavelengths of 500 nm, 540 nm, and 580 nm belong to the band A shown in FIGS. 4 and 5, and the two wave lengths of 620 nm and 660 nm belong to the band B shown in FIGS. 4 and 5.

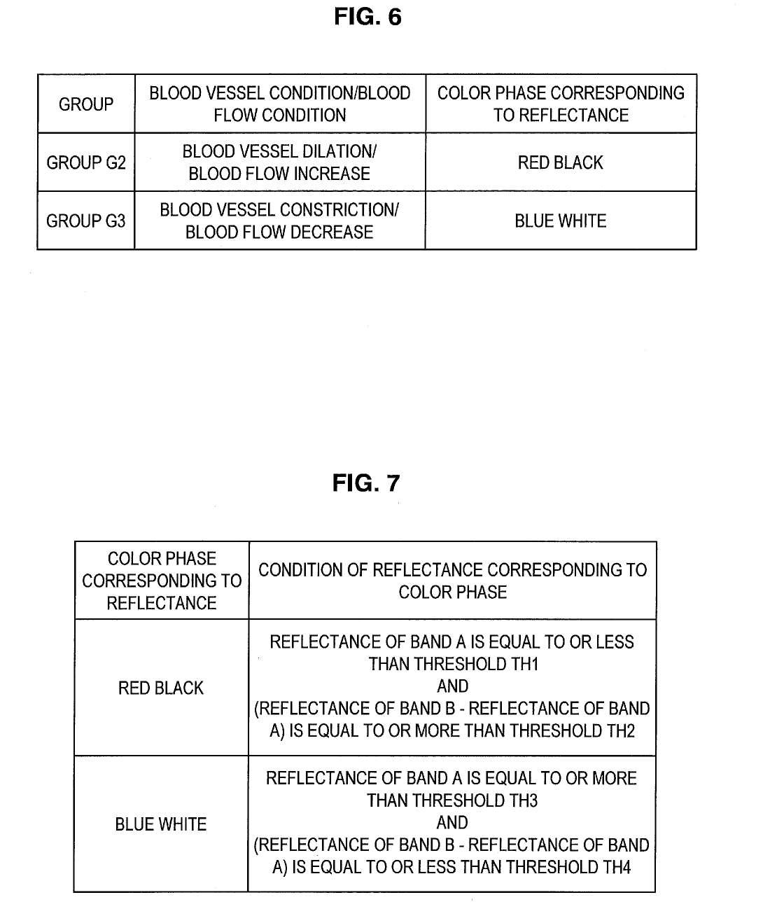

When the inventors focused on the reflectance of the skin of the subjects who belong to the groups G2 and G3 shown in FIG. 1, the reflectance of the skin that belongs to these groups roughly takes a graph form as shown in FIG. 4, and they found out that values of the reflectance have some distinctive points That is, as shown in FIG. 6 that the subjects who belong to the group G2 correspond to a condition of blood vessel dilation/blood flow increase, it was found out that the reflectance of the subjects exhibits a reflectance property corresponding to the color phase “red black”. As also shown that the subjects who belong to the group G3 correspond to a condition of blood vessel constriction/blood flow decrease, it was found out that the reflectance of the subjects exhibits a reflectance property corresponding to the color phase “blue white”.

Here, that the color phase is “red black” corresponds to a condition where the magnitude of the reflectance that belongs to the band A is relatively small within the visible light band of 400 to 700 nm and the magnitude of the reflectance that belongs to the band B is relatively large compared to the reflectance that belongs to the band A. Also, that the color phase is “blue white” corresponds to a condition where the magnitude of the reflectance that belongs to the band A is relatively large in the visible light band of 400 to 700 nm and the magnitude of the reflectance that belongs to the band B is not much different from that of the reflectance that belongs to the band A.

Therefore, according to the relationship shown in FIG. 6, the judgment unit 103 can judge the subject corresponding to the focused measurement data to be in a condition in which the blood vessel is dilated and the blood flow is increased when the color phase of the measurement data output from the measurement data acquisition unit 101 corresponds to “red black”. Meanwhile, the judgment unit 103 can judge the subject corresponding to the focused measurement data to be in a condition in which the blood vessel is constricted and the blood flow is decreased when the color phase of the measurement data output from the measurement data acquisition unit 101 corresponds to “blue white”.

Here, to define the conditions in which the color phases are “red black” and “blue white” more specifically, they can be like shown in FIG. 7.

That is, with reference to the measurement data, the judgment unit 103 specifies the color phase of the reflectance as “red black” and judges that the blood vessel is dilated and the blood flow is increased when the following two conditions (a) and (b) are both established. Meanwhile, with reference to the measurement data, the judgment unit 103 specifies the color phase of the reflectance as “blue while” and judges that the blood vessel is constricted and the blood flow is decreased when the following two conditions (c) and (d) are both established. Also, the judgment unit 103 judges that the blood vessel or the blood flow is in a normal condition when the measurement data does not meet any of the following conditions.
Red Black: Condition of Blood Vessel Dilation/Blood Flow Increase
   (a) Reflectance of Band A is Threshold TH1 or less
   (b) Value of (Reflectance of Band B—Reflectance of Band A) is Threshold TH2 or more
Blue White: Condition of Blood Vessel Constriction/Blood Flow Decrease
   (c) Reflectance of Band A is Threshold TH3 or more
   (d) Value of (Reflectance of Band B—Reflectance of Band A) is Threshold TH4 or less

Here, as a value of the reflectance which is used when judging whether or not the above conditions are established, the judgment unit 103 may use a maximum value, a minimum value, an average value, or the like of all wavelengths which belong to an appropriate band. Alternatively, as a value of the reflectance which is used when judging whether or not the above conditions are established, the judgment unit 103 may also uses a maximum value, a minimum value, an average value, or the like of the reflectance of all of the distinctive wavelengths shown in FIG. 5.

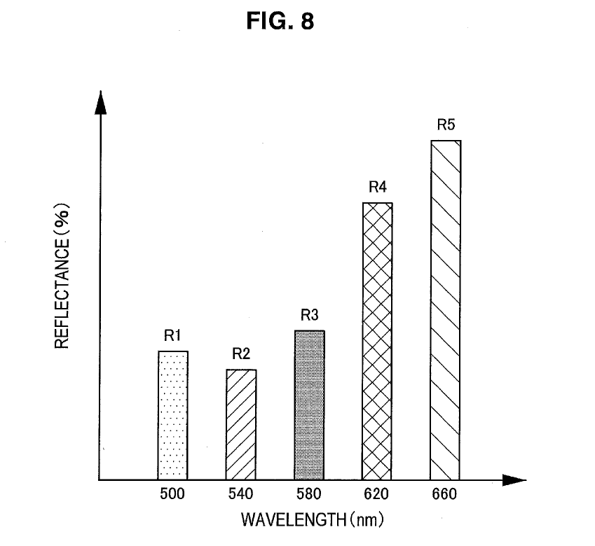

For example, a case is considered in which the judgment unit 103 makes a judgment by using the reflectance R1 to R5 of the five wavelengths shown in FIG. 8. In this case, as described above, the wavelength of 500 nm, the wavelength of 540 nm, and the wavelength of 580 nm belong to the band A, and the wavelength of 620 nm and the wavelength of 660 nm belong to the band B. The judgment unit 103 may use the average value (R1+R2+R3)×(⅓), the maximum value R3, or the minimum value R2 as the reflectance of a wavelength which belongs to the band A. Similarly, the judgment unit 103 may use the average value 0.5×(R4+R5), the maximum value R5, or the minimum value R4 as the reflectance of a wavelength which belongs to the band B.

Here, the above four kinds of thresholds TH1 to TH4 can be respectively determined by measuring the reflectance of the statistically sufficient number of subject groups which belong to the groups G2 and G3 shown in FIG. 1, and by analyzing the obtained measurement result.

Also, since setting values of the above four kinds of thresholds TH1 to TH4 may vary depending on gender of the subjects due to a structural difference in a living body, the setting values of the thresholds TH1 to TH4 may be altered depending on the subject being either a male or a female.

When the inventors analyzed with respect to a group of certain subjects and determined the above-described thresholds, and the like, the following values were obtained, for example. Note that the following specific conditions are just a specific example, and the setting value of a combination of the reflectance and the thresholds used for judging the conditions are not limited to the following example.

### [Male]

Red Black: Condition of Blood Vessel Dilation/Blood Flow Increase
   (a) Reflectance R2 of Wavelength 540 nm≦26%
   (b) (Reflectance R4 of Wavelength 620 nm−Reflectance R3 of Wavelength 580 nm)≧16%
Blue White: Condition of Blood Vessel Constriction/Blood Flow Decrease
   (c) Reflectance R2 of Wavelength 540 nm≦27%
   (d) (Reflectance R4 of Wavelength 620 nm−Reflectance R3 of Wavelength 580 nm)≧15.5%

### [Female]

Red Black: Condition of Blood Vessel Dilation/Blood Flow Increase
   (a) Reflectance R2 of Wavelength 540 nm≦28%
   (b) (Reflectance R5 of Wavelength 660 nm−Reflectance R2 of Wavelength 540 nm)≧23.5%
Blue White: Condition of Blood Vessel Constriction/Blood Flow Decrease
   (c) Reflectance R3 of Wavelength 620 nm≧50%
   (d) (Reflectance R4 of Wavelength 620 nm−Reflectance R3 of Wavelength 580 nm)≧14%

When judging the conditions of the blood vessel or the blood flow of the subject corresponding to the measurement data by the above judgment conditions, the judgment unit 103 according to the present embodiment outputs an obtained judgment result to the concentration calculator 105, the result output unit 107, and the application execution unit 113, which are described later. Also, the judgment unit 103 may store the obtained measurement data as history information in the storage unit 111 in association with time information regarding time and date when the judgment result was generated.

Referring back to FIG. 3, the concentration calculator 105 according to the present embodiment will be described.

The concentration calculator 105 according to the present embodiment is, for example, realized by a CPU, a ROM, and a RAM. The concentration calculator 105 calculates the concentration of a measurement target component at a measurement portion by using the measurement data in relation to the reflectance of light output from the measurement data acquisition unit 101. Examples of the measurement target components, the concentrations of which are calculated by the concentration calculator 105, include various blood components such as hemoglobin like glycosylated hemoglobin, oxygenated hemoglobin, or reduced hemoglobin, and a melanin in the skin. The concentration calculator 105 is capable of calculating the concentration of any component other than the measurement target components as long as the component can be calculated from the reflectance of light at the skin.

When the concentration calculation 105 calculates the concentration of the measurement target component, any known methods for calculating the concentration can be used, and the following method can be used, for example.

Note that, hereinafter, an example will be described in detail in which the concentration calculator 105 calculates the concentrations of four kinds of the measurement target components, namely, a melanin, reduced hemoglobin, oxygenated hemoglobin, and glycosylated hemoglobin by using the measurement data that the measurement data acquisition unit 101 has acquired.

When the measured reflectance is t, the concentration per unit optical path length is cl (unit: mol/L·cm), and molar absorption coefficient is ε, the following formula 101 is established according to Lambert-Beer law,

log(1/t)=ε·cl (Formula 101)

Also, the molar absorption coefficient and the concentration per unit optical path length for a melanin, reduced hemoglobin, oxygenated hemoglobin, and glycosylated hemoglobin are represented as follows:
Melanin
   Molar Absorption Coefficient: ε1, Concentration Per Unit Optical Path Length Mn
Reduced Hemoglobin
   Molar Absorption Coefficient: ε2, Concentration Per Unit Optical Path Length Hb
Oxygenated Hemoglobin
   Molar Absorption Coefficient: ε3, Concentration Per Unit Optical Path Length HbO2
Glycosylated Hemoglobin
   Molar Absorption Coefficient: ε4, Concentration Per Unit Optical Path Length HbAlc

When the reflectance of a certain wavelength in the measurement data is represented as S, and the reflectance of a boundary surface within the human body is represented as D, the following formula 102 is established for each focused wavelength according to the above formula 101.

Mn·ε1+Hb·ε2+HbO2·ε3+HbAlc·ε4+D=−log S (Formula 102)

Therefore, the concentration calculator 105 can obtain a series of simultaneous equations by acquiring the molar absorption coefficient of the measurement target component from the storage unit 111, and the like described later while considering the above formula 102 for each focused wavelength (for example, the five wavelengths shown in FIG. 5). The concentration calculator 105 can calculate the concentration of the measurement target component (that is, the concentration per unit optical path length) by solving the simultaneous equations.

Here, a case is considered in which it is judged by the judgment unit 103 that the subject is in a condition of blood vessel dilation/blood flow increase. In this case, as is clear from the findings of the group G2 in FIG. 1, the concentration of the measurement target component calculated as described above reflects influence of the blood vessel dilation/blood flow increase, thereby being overestimated.

On the other hand, a case is considered in which it is judged by the judgment unit 103 that the subject is a condition of blood vessel constriction/blood flow decrease. In this case, as is clear from the findings of the group G3 in FIG. 1, the concentration of the measurement target component calculated as described above reflects influence of the blood vessel constriction/blood flow decrease, thereby being underestimated.

Therefore, when it is judged by the judgment unit 103 that the subject is in the condition of the blood vessel dilation/blood flow increase, the concentration calculator 105 calculates the concentration after correction by dividing the above calculated concentration by a predetermined correction coefficient α1 to remove the influence of the blood vessel dilation/blood flow increase. Also, when it is judged by the judgment unit 103 that the subject is in the condition of blood vessel constriction/blood flow decrease, the concentration calculator 105 calculates the concentration after correction by multiplying the above calculated concentration by a predetermined correction coefficient a2 to remove the influence of the blood vessel constriction/blood flow decrease.

Here, the correction coefficient used by the concentration calculator 105 for a concentration correction can be determined as follows.

That is, in the scatter diagram as shown in FIG. 1, each plot which belongs to the group G3 is specified, and a value k by which a concentration c is multiplied is calculated for each plot so that the concentration c, which has been calculated based on the reflectance, falls within an allowable limit for error. After the value k to be multiplied is specified for each plot, which belongs to the group G3, a likely correction coefficient is estimated by using the values. The likely correction coefficient is used for causing each plot which belongs to the group G3 to belong to the group G1. By doing such estimation, the correction coefficient a2 for removing the influence of the blood constriction/blood flow decrease can be determined. By carrying out a similar process for each plot which belongs to the group G2, the correction coefficient α1 for removing the influence of the blood constriction/blood flow decrease can be determined.

When the inventors analyzed with respect to a group of certain subjects, and determined the correction coefficients, values α1=α2=1.3 were obtained. Note that the correction coefficients are just a specific example, and the coefficient used for the concentration correction process is not limited to the above values.

The concentration calculator 105 calculates the concentration of the measurement target component as described above, carries out the concentration correction process as necessary, and outputs an obtained calculated result of the concentration to the result output unit 107 described later. Also, the concentration calculator 105 may store the obtained calculated result of the concentration as history information in the storage unit 111 in association with time information regarding time and date when the concentration is calculated.

The result output unit 107 is realized by a CPU, a ROM, a RAM, an output unit, a communication unit, and the like. The result output unit 107 outputs, to the user of the information processing apparatus 10, the judgment result of the condition of the blood vessel or the blood flow output from the judgment unit 103, or the calculation result of the concentration of the measurement target component output from the concentration calculator 105.

For example, the result output unit 107 outputs the judgment result by the judgment unit 103 or the calculation result of the concentration by the concentration calculator 105 to a display unit such as a display via the display controller 109 described later. Also, the result output unit 107 may output the judgment result by the judgment unit 103 or the calculation result of the concentration by the concentration calculator 105 to other devices provided outside the information processing apparatus 10 via various networks, or the like. Also, the result output unit 107 may output the judgment result by the judgment unit 103 or the calculation result of the concentration by the concentration calculator 105 as printed material via the output unit such as a printer.

The display controller 109 is realized by a CPU, a ROM, a RAM, an output unit, a communication unit, and the like. The display controller 109 controls a display screen of the display unit such as a display provided to the information processing apparatus 10, or of a display unit such as a display provided outside the information processing apparatus 10. To be more specific, the display controller 109 controls the display screen based on information relating to the judgment result by the judgment unit 103 or the calculation result of the concentration by the concentration calculator 105. The display controller 109 carries out the display control of the processed results to the display screen, whereby the user of the information processing apparatus 10 can get the judgment result by the judgment unit 103 or the calculation result of the concentration by the concentration calculator 105.

The storage unit 111 is realized by a RAM, a storage unit, or the like provided in the information processing apparatus 10 of the present embodiment. The storage unit 111 stores various information such as the molar absorption coefficient or a color pattern of the measurement target component used by the concentration calculator 105 for the concentration calculation process, and execution data for various applications executed by the application execution unit 113 described later. Also, the storage unit 111 properly stores various parameters or progress of a process which need to be saved when the information processing apparatus 10 executes some sort of process, various databases, and the like. The storage unit 111 has a structure to/from which each processing unit provided in the information processing apparatus 10 of the present embodiment is capable of freely reading/writing.

The application execution unit 113 is realized by a CPU, a ROM, a RAM, a communication unit, or the like. The application execution unit 113 provides various services to the user of the information processing apparatus 10 by executing an application which uses the judgment result by the judgment unit 103 or the calculation result of the concentration by the concentration calculator 105.

To be more specific, the application execution unit 113 executes the application which supports health management of the user of the information processing apparatus 10 by displaying development of the judgment result by the judgment unit 103 or the calculation result of the concentration by the concentration calculator 105 over time. The information processing apparatus 10 according to the present embodiment can easily judge the conditions of the blood vessel or the blood flow based on the reflectance, whereby the judgment result itself can be used as an index which contributes to the user's health management, or the like. Also, the concentration of a component from which the influence of the conditions of the blood vessel or the blood flow is removed can be calculated, whereby it is possible to provide a service for supporting the health management using more correct values.

Also, the application execution unit 113 can execute an application for judging whether or not there is stiffness in the neck, the shoulders, the waist, or the like by using the judgment result by the judgment unit 103 or the calculation result of the concentration by the concentration calculator 105. That is, the information processing apparatus 10 of the present embodiment is capable of specifying a condition of the blood flow and a high/low concentration of hemoglobin. Here, in a case where the blood flow is decreased and the concentration of hemoglobin is low, it can be judged that the subject is in a condition where metabolism slows down and waste is liable to build up in the muscles. Therefore, in that case, it is possible to notify the user of the information processing apparatus 10 that the body is likely to suffer the stiffness. Also, in a case where the blood flow is increased and the concentration of hemoglobin is high, it can be considered that the subject has good metabolism, and therefore, it is possible to notify the user to that effect.

Also, the application execution unit 113 is capable of specifying excitement of the user by using the judgment result by the judgment unit 103 or the calculation result of the concentration by the concentration calculator 105, and reflects the specified excitement in an execution parameter of the application. That is, since the information processing apparatus 10 of the embodiment can acquire sharp increase/decrease of the blood flow, it is possible to judge whether or not the user is in an excited condition by periodically measuring a change of the blood flow. The judgment result can be used as an execution parameter of an application such as a game, or can be presented to the user, for example.

Note that the above specific example of the applications is just an example, and the application that the application execution unit 113 according to the present embodiment executes is not limited to the above example.

Also, the functions of the above measurement data acquisition unit 101, the judgment unit 103, the concentration calculator 105, the result output unit 107, the display controller 109, the storage unit 111, and the application execution unit 113 can be incorporated into any hardware as long as each hardware can transmit/receive information to/from each other via a network. Also, a process carried out by a processing unit may be realized by a single unit of hardware, or may be realized in distributed processing by multiple units of hardware.

As described above, an example of the function of the information processing apparatus 10 according to the present embodiment has been described. Each of the above configuration elements may be configured from a general-purpose component or circuit, or may be configured from hardware which is specialized for a specific function of each of the configuration elements. Also, all of the functions of each of the configuration elements may be performed by a CPU, or the like. Therefore, a hardware configuration for use can be properly altered depending on a technology level of the time when the present embodiment is implemented.

Note that, it is possible to fabricate a computer program for realizing each of the functions of the information processing apparatus according to the above-described present embodiment, and to incorporate the computer program into a personal computer, or the like. Further, it is possible to provide a computer-readable recording medium in which the computer program is stored. The recording medium can be a magnetic disk, an optical disk, a magneto optical disk, a flash memory, or the like. Also, the above computer program may be distributed via a network, for example, instead of using the recording medium.

### <Example of Measurement Unit>

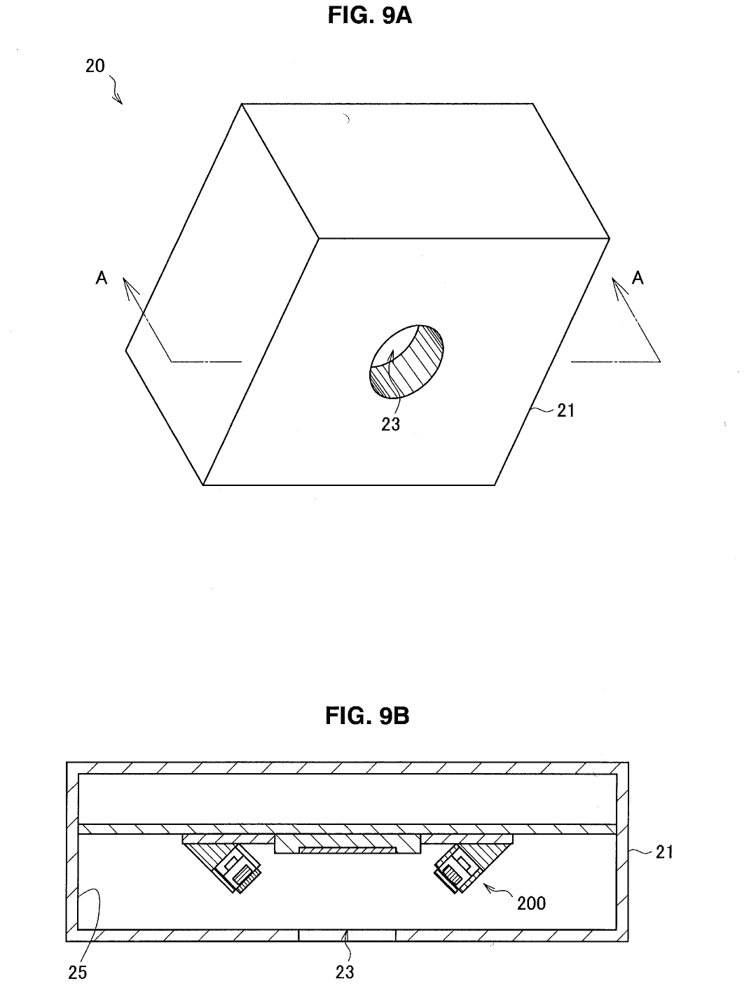

Next, an example of the measurement unit 20 according to the present embodiment will be described with reference to FIGS. 9A and 9B. FIGS. 9A and 9B are explanatory diagrams schematically showing an entire configuration of the measurement unit 20 of the present embodiment.

### [Entire Configuration of Measurement Unit]

The measurement unit 20 according to the present embodiment, as shown in FIG. 9A, has a case 21 made from any selected material, and an opening 23 is provided at a portion of the case 21. In FIG. 9A, the shape of the opening 23 is a circle; however, the shape of the opening 23 is not limited to the circle, and may be a polygon or an ellipse. A measurement target object (for example, a skin surface of a human body) is placed on the opening 23, and the measurement unit 20 of the present embodiment carries out measurement with respect to the placed measurement target object. Note that the size of a through hole of the opening 23 can be properly determined in accordance with the size of a light-receiving element that an optical component 200 described later includes.

FIG. 9B is a sectional view showing a section of FIG. 9A taken along a cutting line A-A.

As shown in FIG. 9B, the inside of the case 21 is hollow, and the optical component 200 of the measurement unit 20 of the present embodiment is mounted the inside the case 21. Also, it is preferable that an inner wall of the case 21 be black or a dark-tone color close to black in order to reduce reflection of light leaked from the optical component 200.

Here, the optical component 200 mounted in the case 21 will be described in detail later again. Also, in FIG. 9B, only the optical component 200 is illustrated inside the case 21; however, any unit other than the optical component 200 can be mounted inside the case 21 as long as the unit does not affect a measurement process in the optical component 200.

### [Optical Component Configuration]

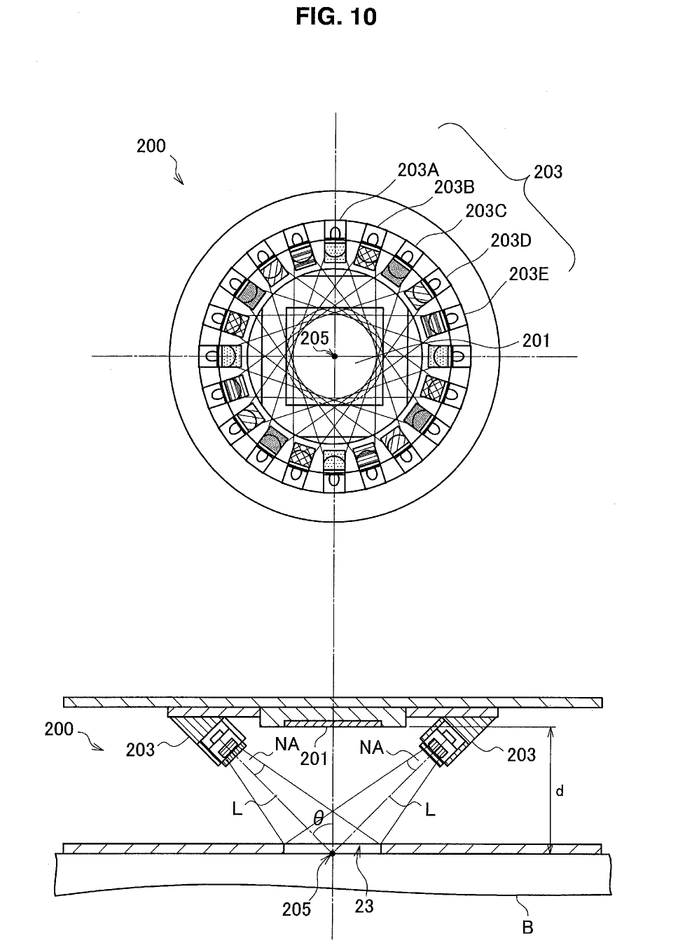

Next, an optical component that the measurement unit 20 of the present embodiment includes will be described in detail with reference to FIG. 10. FIG. 10 is an explanatory diagram schematically showing an example of an optical component that the measurement unit of the present embodiment includes.

The upper diagram of FIG. 10 is a plan view when the optical component 200 of the present embodiment is seen from a side of the opening 23, and the lower diagram of FIG. 10 is a sectional view when the optical component 200 of the present embodiment is cut by the central line of the upper diagram of FIG. 10. Note that, in the example shown in FIG. 10, a case will be described where a skin surface B of a human body is placed on the opening 23, and the skin surface B placed on the opening 23 is a measurement target region.

As shown in FIG. 10, the optical component 200 of the present embodiment includes a light-receiving element 201 arranged in a container unit having an arbitrary shape like a substrate, and a plurality of light-emitting elements 203 arranged in a container unit having an arbitrary shape like a substrate.

Light (reflected light) from the measurement target region on which the measurement target region is placed is imaged on the light-receiving element 201. The light-receiving element 201 generates data which represents an optical amount of imaged light in accordance with an optical amount of the light imaged on a receiving surface. An example of the light-receiving element 201 includes a photodiode; however, the light-receiving element 201 of the present embodiment is not limited to the photodiode, and other optical sensors can be used. Also, the light-receiving element 201 may measure other physical amounts such as a luminance value of the imaged light instead of measuring the optical amount of the light imaged on the receiving surface.

The light-receiving element 201 is, as shown in FIG. 10, arranged to face the opening 23 provided in the case 21 of the measurement unit 20. In other words, the light-receiving element 201 is arranged to face the opening 23 approximately in parallel thereto. Also, the size of the light-receiving element 201 can be properly determined in accordance with the through hole provided as the opening 23, and for example, a small optical sensor having a size of 10 mm×10 mm, or the like can be used. When such a small optical sensor is used, it is preferable that the size of the opening 23 be 10 mmφ, for example.

As shown in the upper diagram of FIG. 10, the plurality of light-emitting elements 203 is arranged on the periphery of the light-receiving element 201 in a circular manner. As the light-emitting element 203, a light-emitting diode (LED) can be used.

The light-emitting elements 203 are evenly arranged at a regular interval around a center 205 of the opening 23. For example, 4N light-emitting elements (N is an integer of 1 or more) 203 are arranged at an interval of (90/N)° around the center 205 of the opening 23. The number of the light-emitting elements 203 arranged around the light-receiving element 201 can be properly set in accordance with the size of the receiving light-emitting element 201, the size of the measurement unit 20 itself, or the like, but it is preferable to arrange twenty light-emitting elements at the interval of 18°.

Also, a wavelength of light irradiated from the light-emitting element 203 can be properly selected depending on which characteristic of the measurement target object is measured; however, it is preferable to use the light-emitting element which is capable of irradiating with light of within the visible light band (about 400 to 700 nm), for example.

The plurality of the light-emitting elements 203 is, as shown in the lower diagram of FIG. 10, arranged to incline with respect to the normal line of the measurement target region so that a central line L of irradiated light from each light-emitting element 203 passes through the center 205 of the measurement target region. Also, it is preferable that the size of a spot of the irradiated light from each light-emitting element 203 at the measurement target region be approximately the same as (be roughly overlapped with) the size of the opening 23, as shown in the lower diagram of FIG. 10. Note that, in the lower diagram of FIG. 10, an angle formed by the central line L of the irradiated light and the normal line of the measurement target region is represented as θ. Hereinafter, the angle θ will be referred to as an arranged angle of the light-emitting element 203.

The arranged angle of the light-emitting element 203 is set in accordance with an offset distance between the measurement target region and the light-receiving element 201 (the distance d in the lower diagram of FIG. 10). That is, when the offset distance d is a predetermined threshold or less (for example, 20 mm), the arranged angle θ can be 45°, and when the offset distance d is more than a predetermined threshold (for example, 20 mm), the arranged angle θ can be less than 45° (preferably, 20 to 30°). The arranged angle θ can be determined for each of the focused wavelengths based on the reflectance measured by using the integrating sphere by specifying an angle, by which reflectance roughly equivalent to the reflectance obtained by the integrating sphere can be obtained.

Note that the offset distance between the measurement target region and the light-receiving element 201 can be set to be any large value as long as downsizing of the measurement unit is not aimed; however, it is preferable that an upper limit of the offset distance d be about 40 mm in the case of the measurement unit 20 of the present embodiment.

Also, it is preferable that the light-emitting element 203 of the measurement unit 20 of the present embodiment be capable of irradiating with light of a low numerical aperture. The light of the low numerical aperture may be realized by irradiation from the light-emitting element 203 itself, or may also be realized by combining the light-emitting element with a predetermined condenser lens. The numerical aperture NA of the irradiated light from the light-emitting element 203 can be set by making a graph which indicates dependency of the arranged angle of the reflectance, and causing a curve line which indicates the dependency of the arranged angle to be intersected with a measurement result of the reflectance measured by the integrating sphere. By determining the numerical aperture NA in this way, the curve line which indicates the dependency of the arranged angle of the reflectance becomes intersected with the measurement result of the reflectance measured by the integrating sphere, and it is possible to determine a proper arranged angle θ. Note that a specific value of the numerical aperture NA can be properly set in accordance with the size of the measurement unit 20, or the like; however, it is desirable to be about 0.2 to 0.3, for example.

Here, the measurement unit 20 of the present embodiment effectively measures the skin of a human by irradiating with light of the five kinds of wavelengths shown in FIG. 5, for example. Note that the five kinds of wavelengths shown in FIG. 5 are useful when various hemoglobin such as oxygenated hemoglobin, glycosylated hemoglobin, and reduced hemoglobin existing in the blood of the human are a measurement target.

As described above, in the optical component 200 of the measurement unit 20 of the present embodiment, the measurement target region is irradiated with N kinds of wavelengths from the 4N light-emitting elements 203. It is desirable to irradiate the measurement target region with light by delaying irradiation timing of the N kinds of wavelengths. When the light-emitting element 203 irradiates with light of a predetermined wavelength by a single pulse waveform being input to the light-emitting element 203, it is desirable that the N kinds of wavelengths be irradiated by N pulse waveforms by time division. In this case, to ensure an optical amount sufficient for measurement by a single irradiation, it is desirable to set a width of the pulse waveform to be 1 millisecond (ms) or more for each wavelength λN. Also, to prevent color mixture of light of different wavelengths, it is desirable that a temporal position of the pulse waveform of a t^{th} wavelength λt and a temporal position of the pulse waveform of a t+1^{th} wavelength λt+1 be 2 milliseconds or more.

By controlling irradiation by the time division as described above, the measurement target region is sequentially irradiated with the N kinds of wavelengths, and reflected light of each wavelength is imaged at the light-receiving element 201. As a result, it becomes possible to properly measure the optical amount of the reflected light corresponding to each wavelength at the light-receiving element 201.

As described above, the measurement unit 20 of the present embodiment can obtain optical information of the measurement target object placed on the measurement target region by sequentially irradiating the measurement target region with light of the N kinds of wavelengths, and by measuring reflected light corresponding to each wavelength by the light-receiving element. Also, since a distinctive wavelength of a phenomenon or an object to be measured is selected in advance before carrying out measurement, it is not necessary for the measurement unit 20 of the present embodiment to have an optical unit such as the integrating sphere or a diffraction grating, whereby downsizing of the apparatus can be realized. Also, since a light irradiating diode can be used as a light source of the N kinds of wavelength, even when the 4N light-emitting elements are mounted, a lower cost and higher power saving performance can be achieved.

Note that the measurement unit 20 shown in FIGS. 9A to 10 irradiates the measurement target region with light of the N kinds of wavelengths by different timing, and measures the measurement target object placed on the measurement target region. A first modification of the measurement unit 20 described below has 4N light-emitting elements which simultaneously irradiate the measurement target region with light of the same wavelength, and measures the measurement target object placed on the measurement target region. At this time, the measurement unit 20 selects focused N kinds of wavelengths by arranging an optical filter right before the light-receiving element 201.

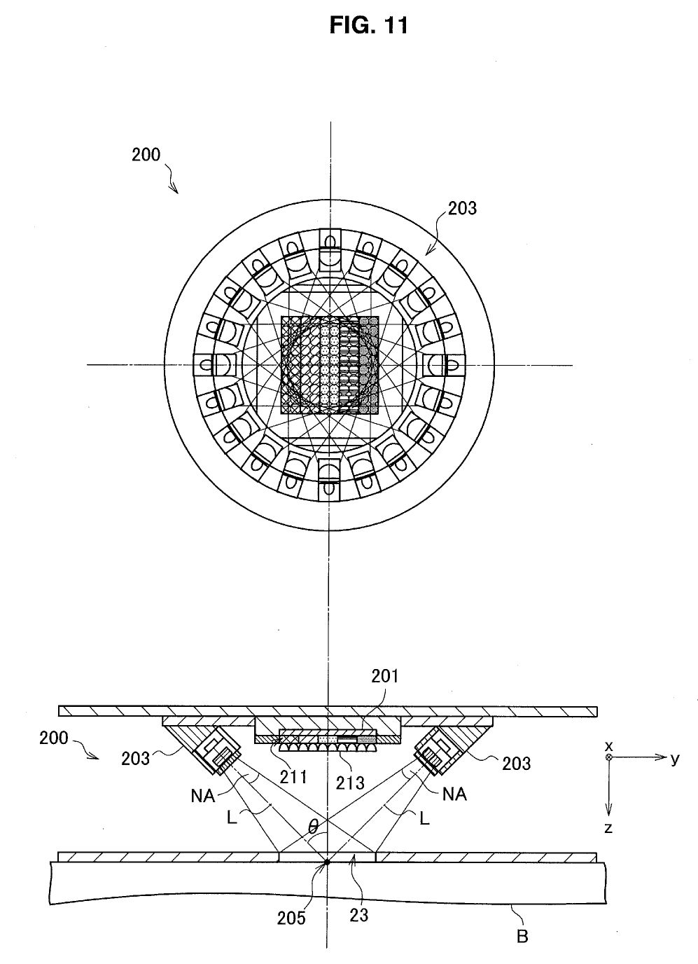

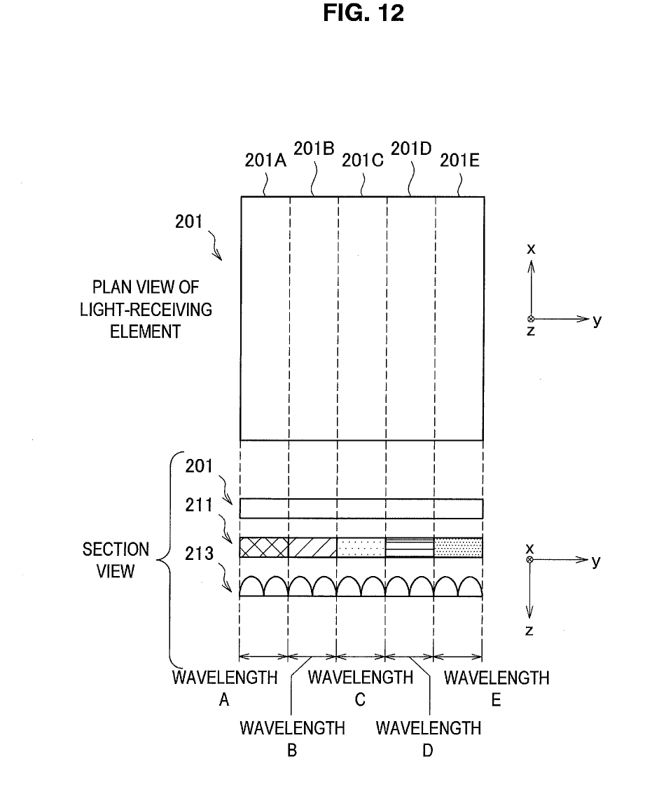

First of all, hereinafter, a configuration of an optical component 200 provided in a measurement unit 20 according to the present modification will be described in detail with reference to FIG. 11. FIG. 11 is an explanatory diagram schematically showing an example of an optical component included in a measurement unit according to the present modification.

The upper diagram of FIG. 11 is a plan view when the optical component 200 of the present modification is seen from an opening 23, and the lower diagram of FIG. 11 is a sectional view when the optical component 200 of the present modification is cut by the central line of the upper diagram of FIG. 11. Note that, in the example shown in FIG. 11, a case will be described in which a skin surface of a human body is placed on the opening 23, and the skin surface placed on the opening 23 is a measurement target region.

The optical component 200 of the present modification includes, as shown in FIG. 11, a light-receiving element 201 arranged in a container unit having an arbitrary shape like a substrate, and a plurality of light-emitting elements 203 arranged in a container unit having an arbitrary shape like a substrate. Also, at an upper portion of a receiving surface of the light-receiving element 201 (in the z-axis forward direction in the lower diagram of FIG. 11), an optical filter 211 and a collimator lens 213 are provided.

Light transmitted through the collimator lens 213 and the optical filter 211 is imaged on the light-receiving element 201 from among reflected light from the measurement target object placed on the measurement target region. The light-receiving element 201 generates data which represents an optical amount of imaged light in accordance with an optical amount of the light imaged on a receiving surface. An example of the light-receiving element 201 includes a photodiode; however, the light-receiving element 201 of the present modification is not limited to the photodiode, and other optical sensors can be used.

The light-receiving element 201 is arranged to face the opening 23 provided in the case 21 of the measurement unit 20.

As shown in the upper diagram of FIG. 11, a plurality of light-emitting elements 203 having the same irradiation property is arranged on the periphery of the light-receiving element 201 in a circular manner. As the light-emitting element 203, similar to the above description, a light-emitting diode can be used, for example.

The light-emitting elements 203 are evenly arranged at a regular interval around a center 205 of the opening 23. For example, 4N light-emitting elements (N is an integer of 1 or more) 203 are arranged at an interval of (90/N)° around the center 205 of the opening 23. The number of the light-emitting elements 203 arranged around the light-receiving element 201 can be properly set in accordance with the size of the light-receiving element 201 or the measurement unit 20 itself, or the like, but it is desirable to arrange twenty light-emitting elements at the interval of 18°.

Also, a wavelength of light irradiated from the light-emitting element 203 can be properly selected in accordance with which characteristic of the measurement target object is measured; however, it is desirable to use a light-emitting element 203 which is capable of irradiating with light of within a wavelength band which covers a distinctive wavelength of a target subject. By using a white light-emitting element as the light-emitting element 203 of the present modification, for example, a measurement target having a distinctive wavelength which exists within the visible light band (about 400 to 700 nm) can be measured.

The plurality of the light-emitting elements 203 is, as shown in the lower diagram of FIG. 11, arranged to incline with respect to the normal line of the measurement target region so that the central line L of irradiated light from each light-emitting element 203 passes through the center 205 of the measurement target region. Also, it is desirable that, as shown in the lower diagram of FIG. 11, the size of a spot of the irradiated light from each light-emitting element 203 at the measurement target region be approximately the same as (be roughly overlapped with) the size of the opening 23.

Note that, it is preferable that an arranged angle θ and a numerical aperture NA of the light-emitting element 203 be set to values fallen within a similar range to the above described case.

The plurality of the light-emitting elements 203 irradiates with light having the same wavelength property as described above, and it is preferable that the plurality of the light-emitting elements 203 simultaneously irradiate with the light. Therefore, when the light-emitting element 203 irradiates with white light by a pulse waveform being input to the light-emitting element 203, the plurality of the light-emitting elements 203 is simultaneously irradiated by N pulse waveforms being simultaneously input to the plurality of the light-emitting elements 203. In this case, to ensure an optical amount sufficient for measurement by a single irradiation, it is preferable to set a width of the pulse waveform to be 1 millisecond (ms) or more.

Here, in the measurement unit 20 of the present modification, a measurement process is carried out by focusing on a distinctive wavelength of a measurement target (for example, a skin surface of a human). In the following description, there are N distinctive wavelengths for the measurement target. In this case, the optical component 200 of the measurement unit 20 of the present modification measures, for the N distinctive wavelengths, an optical amount by using light reflected on a surface of the measurement target, the light being irradiated from the light-emitting element 203 having the same wavelength property (for example, irradiating with white light).

The optical component 200 of the present modification uses an optical filter 211 as shown in the lower diagram of FIG. 11 in order to select light of a focused wavelength from the reflected light using a white light source. The optical filter 211 is provided at an upper portion of a receiving surface of the light-receiving element 201 by the number of focused wavelengths. In the present modification, N kinds of the optical filters 211 are used since N kinds of distinctive wavelengths are focused.

The optical filter 211 is, as described above, an optical element (for example, bandpass filter) which transmits light of within a specific wavelength band. In the optical component 200 of the present modification, it is possible to properly select the optical filter 211 in accordance with the focused wavelength band as the distinctive wavelength of the measurement target. Since the N kinds of wavelengths are focused in the present modification, N kinds of the optical filters 211 which respectively transmit the N kinds of wavelengths are selected.

Here, in relation to each optical filter 211, a wavelength band of the light transmitted through the filter can be properly set in accordance with a property of the focused wavelength.

Reflected light transmitted through the optical filter 211 is imaged on a specific region of the light-receiving element 201. Therefore, the measurement unit 20 of the present modification can specify to which wavelength band light imaged on a specific region of the light-receiving element 201 corresponds, by grasping a positional relationship between the light-receiving element 201 and each of the plurality of the optical filters 211 in advance.

Also, to effectively introduce the reflected light (diffused reflected light) from the measurement target surface into the optical filter 211, a collimator lens 213 such as a rod lens can be arranged at an upper side of the optical filter 211 (in the z-axis forward direction in the lower diagram of FIG. 11). The diffused reflected light that has entered the collimator lens 213 becomes parallel by the collimator lens 213, and enters the optical filter 211.

Next, the optical component 200 of the present modification, especially, the light-receiving element 201 and the optical filter 211 will be described in detail, with reference to an example similar to the above-described case in which a skin surface of a human is set to be the measurement target.

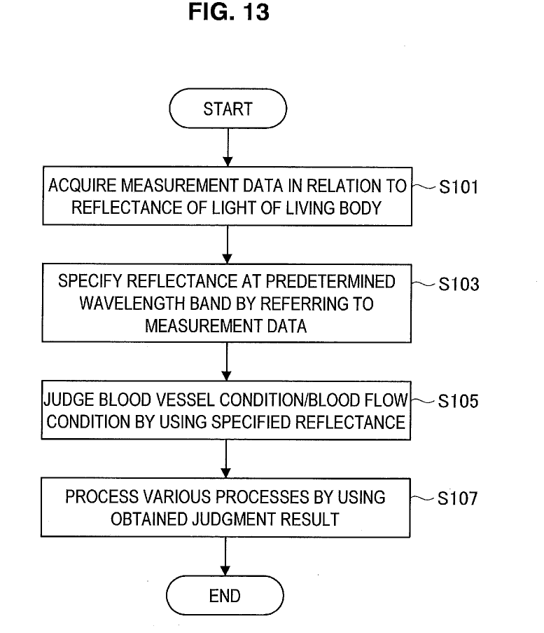

As described in FIGS. 4 and 5, distinctive wavelengths of reflected light from the surface of the human are the five kinds of wavelengths of 500 nm, 540 nm, 580 nm, 620 nm, and 660 nm. Here, to measure an optical amount of light of the five kinds of wavelengths, the light-receiving element 201 is divided into five regions 201A to 201E as shown in the upper diagram of FIG. 12. The five regions may be physically divided at the light-receiving element 201, or may be virtually divided into five regions (due to reasons for processing). Among the five regions of the light-receiving element 201, for example, the region 201A is a region where light of a wavelength A is imaged, and the region 201B is a region where light of a wavelength B is imaged. Here, when a photodiode having a size of 10 mm×10 mm is used as the light-receiving element 201, the photodiode can be divided into five regions each having the size of 2×10 mm.

Note that, in the upper diagram of FIG. 12, a case is shown where a photodiode used as the light-receiving element 201 is evenly divided into five rectangular regions; however, the shape of the region is not limited to rectangular as shown in FIG. 12.

To image light of wavelengths corresponding to the five kinds of receiving regions, as shown in FIG. 12, five kinds of bandpass filters are used as the optical filter 211. In the following description, in order, the wavelength A corresponds to the center wavelength of 500 nm, the wavelength B corresponds to the center wavelength of 540 nm, the wavelength C corresponds to the center wavelength of 580 nm, the wavelength D corresponds to the center wavelength of 620 nm, and the wavelength E corresponds to the center wavelength of 660 nm. Note that the order is for descriptive purpose, and it can be properly determined which region corresponds to which center wavelength.

The five kinds of the optical filters 211 are arranged at an upper portion of a region facing the receiving surface (for example, right above the receiving surface) as shown in the lower diagrams of FIGS. 11 and 12. According to this arrangement, for example, only a predetermined range of light having the center wavelength of 500 nm can be transmitted through the optical filter 211 provided right above the region 201A among from white reflected light which has entered the optical filter 211, whereby the predetermined range of light having the center wavelength of 500 nm is imaged on the region 201A. Note that, in FIG. 12, a gap is provided between the light-receiving element 201 and the optical filter 211 for the purpose of illustration; however, the optical filter 211 may be provided right above the light-receiving element 201, or may be provided with the gap as shown in the drawing.

Also, at an upper portion of the optical filter 211, as described above, a collimator lens 213 for collimating defused reflected light is properly provided.

As described above, the measurement unit 20 of the present modification has been described in detail with reference to FIGS. 11 and 12. In the measurement unit 20 of the present modification, optical information of the measurement target placed on the measurement target region can be obtained by simultaneously irradiating the measurement target region with light of the same wavelength property, and by selecting the wavelength by the optical filter provided at a front stage of the light-receiving element. Also, since a distinctive wavelength of a phenomenon or an object to be measured is selected in advance before carrying out measurement, it is not necessary for the measurement unit 20 to have an optical unit such as the integrating sphere or a diffraction grating, whereby downsizing of the apparatus can be realized. Also, since a light-emitting diode can be used as a light-emitting element, even when the 4N light-emitting elements are mounted, a lower cost and higher power saving performance can be achieved.

### <Information Processing Method>

Next, a flow example of an information processing method implemented in the information processing apparatus 10 according to the present embodiment will be described with reference to FIG. 13. FIG. 13 is a flow diagram showing a flow example of an information processing method according to the present embodiment.

The measurement data acquisition unit 101 of the information processing apparatus 10 according to the present embodiment acquires, from the measurement unit 20, measurement data in relation to reflectance of light of a living body which is measured by the measurement unit 20 (step S101). Then, the measurement data acquisition unit 101 outputs the acquired measurement data to the judgment unit 103.

The judgment unit 103 refers to the measurement data output from the measurement data acquisition unit 101, and specifies reflectance at predetermined wavelength bands (for example, the band of entire 400 to 700 nm, or the five kinds of wavelengths shown in FIG. 5) (step S103). Then, the judgment unit 103 judges a condition of a blood vessel or blood flow by judging whether or not the above conditions are established by using the specified reflectance (step S105).

The judgment unit 103 outputs the judgment result relating to the condition of the blood vessel or the blood flow to the concentration calculator 105 and the application execution unit 113. The concentration calculator 105 and the application execution unit 113 carry out various processes by using the judgment result obtained from the judgment unit 103 (step S107).

For example, the concentration calculator 105 calculates a concentration of the measurement target component such as various kinds of hemoglobin, or a melanin by using the measurement data acquired by the measurement data acquisition unit 101. At this time, in a case where the judgment result by the judgment unit 103 shows dilation of the blood vessel or increase of the blood flow, or in a case where the judgment result by the judgment unit 103 shows constriction of the blood vessel or decrease of the blood flow, the concentration calculator 105 corrects the calculated concentration of the measurement target component, and obtains a concentration after correction as an estimate concentration of the measurement target component.

Further, the application execution unit 113 may judge stiffness of the neck, the shoulders, the waist, or the like by using the judgment result by the judgment unit 103, or may specify and apply a level of excitement of the user to a game by using the judgment result by the judgment unit 103.

As described above, the information processing method according to the present embodiment can easily judge the condition of the blood vessel or the blood flow of the subject based on the measurement result of the reflectance of light at the skin, and the obtained judgment result can be used for various processes.

### (Hardware Configuration)

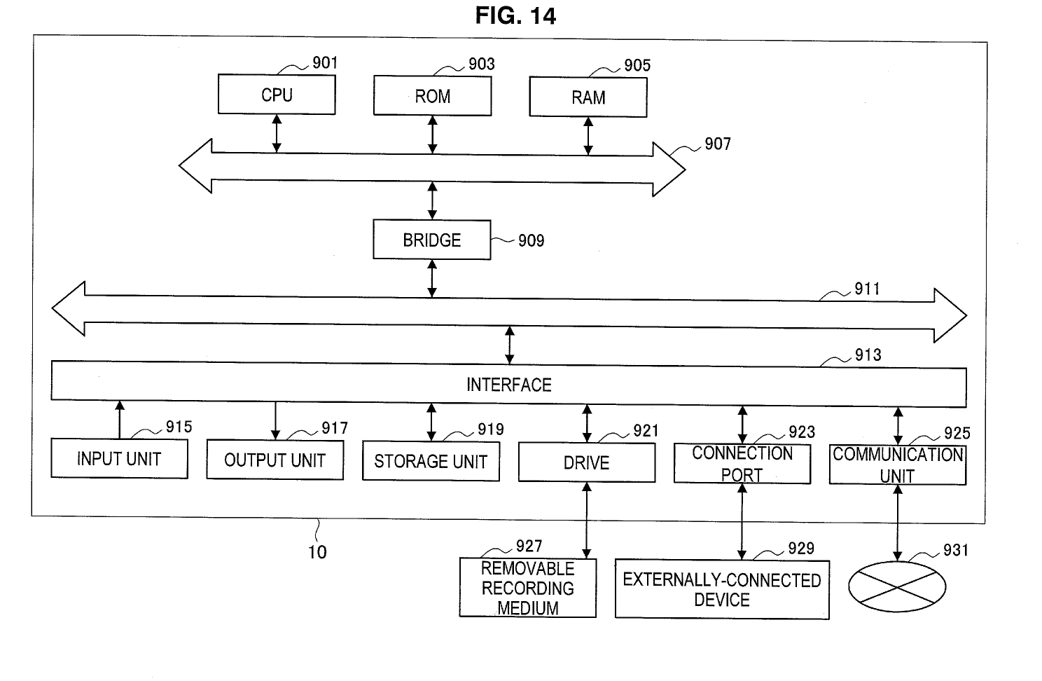

Next, a hardware configuration of the information processing apparatus 10 according to the embodiment of the present disclosure will be described in detail with reference to FIG. 14. FIG. 14 is a block diagram illustrating a hardware configuration of the information processing apparatus 10 according to the embodiment of the present disclosure.

The information processing apparatus 10 principally includes a CPU 901, a ROM 903, and a RAM 905. Further, the information processing apparatus 10 includes a host bus 907, a bridge 909, an external bus 911, an interface 913, an input unit 915, an output unit 917, a storage unit 919, a drive 921, a connection port 923, and a communication unit 925.

The CPU 901 functions as a processing unit and a control unit, and controls overall operation or a part of the operation in the information processing apparatus 10 according to various programs recorded in the ROM 903, the RAM 905, the storage unit 919, or a removable recording medium 927. The ROM 903 stores a program, an operation parameter, and the like that the CPU 901 uses. The RAM 905 temporarily stores a program that the CPU 901 uses, or a parameter and the like which properly vary upon execution of the program. These components are mutually connected by the host bus 907 configured from an internal bus such as a CPU bus.

The host bus 907 is connected to the external bus 911 such as a PCI (Peripheral Component Interconnect/Interface) bus via the bridge 909.

The input unit 915 is operation means that the user operates such as a mouse, a keyboard, a touch panel, a button, a switch, or a lever. Also, the input unit 915 may be remote control means (so-called, remote) that uses infrared light or other radio waves, for example. The input unit 915 may also be an externally-connected device 929 such as a mobile phone or a PDA which supports operation for the information processing apparatus 10. Further, the input unit 915 is configured from an input control circuit, and the like which generate an input signal based on information input by the user with the above-described operation means, and output the signal to the CPU 901. The user of the information processing apparatus 10 can input various data, or can instruct processing operation to the information processing apparatus 10 by operating the input unit 915.

The output unit 917 is configured from a device which is capable of visually or aurally announcing obtained information to the user. Examples of the device include a display unit such as a CRT display unit, a crystal display unit, a plasma display unit, an EL display unit, and LAMP, an audio output unit such as a speaker and a headphone, a printer unit, a mobile phone, and a facsimile machine. The output unit 917 outputs, for example, a result which is obtained from various processing carried out by the information processing apparatus 10. To be more specific, the display unit displays the result obtained from the various processing carried out by the information processing apparatus 10 as a text or an image. Meanwhile, the audio output unit converts an audio signal which includes reproduced audio data, sound data, or the like into an analog signal, and outputs the analog signal.

The storage unit 919 is a device for data storage configured as an example of a memory unit of the information processing apparatus 10. The storage unit 919 is configured from a magnetic memory device such as an HDD (Hard Disk Drive), a semiconductor memory device, an optical memory device, a magneto optical memory device, or the like. The storage unit 919 stores a program or various data that the CPU 901 executes, and various data obtained from outside.

The drive 921 is a reader/writer for recording media, and is accommodated in or is externally-connected to the information processing apparatus 10. The drive 921 reads information recorded on a mounted removable recording medium 927 such as a magnetic disk, an optical disk, a magneto optical disk, or a semiconductor memory, and outputs the information to the RAM 905. Further, the drive 921 is also capable of writing a record on the mounted removable recording medium 927 such as the magnetic disk, the optical disk, the magneto optical disk, or the semiconductor memory. The removable recording medium 927 is a DVD medium, an HD-DVD medium, or a Blu-ray medium, for example. Also, the removable recording medium 927 may be a CompactFlash: CF (registered trademark), a flash memory, an SD memory card (Secure Digital memory card), or the like. The removable recording medium 927 may also be an IC card (Integrated Circuit card) with a contactless IC chip mounted thereon, an electronic device, or the like.

The connection port 923 is a port which directly connects devices to the information processing apparatus 10. Examples of the connection port 923 include a USB (Universal Serial Bus) port, an IEEE1394 port, and a SCSI (Small Computer System Interface) port. Other examples of the connection port 923 include an RS-232C port, an optical audio terminal, and an HDMI (High-Definition Multimedia Interface) port. By connecting the externally-connected device 929 to the connection port 923, the information processing apparatus 10 can directly obtain various data from the externally-connected device 929, or can provide various data to the externally-connected device 929.

The communication unit 925 is a communication interface configured from a communication device, or the like for connecting to a communication network 931. The communication unit 925 may be, for example, a communication card for a wired or wireless LAN (Local Area Network), a Bluetooth (registered trademark), or a WUSB (Wireless USB). Also, the communication unit 925 may be a router for optical communication, a router for an ADSL (Asymmetric Digital Subscriber Line), a modem for various communications, or the like. The communication unit 925 is capable of transmitting/receiving a signal, or the like to/from the Internet, or other communication devices in accordance with a predetermined protocol such as TCP/IP. Also, the communication network 931 connected to the communication unit 925 is configured from a network, or the like connected by wired/wireless connection, and for example, may be the Internet, a home LAN, an infrared communication, a radio wave communication, or a satellite communication.

As described above, an example of the hardware configuration has been shown, which is capable of achieving the function of the information processing apparatus 10 according to the embodiment of the present disclosure. Each of the above configuration elements may be configured from a general-purpose component, or may be configured from hardware which is specialized for a specific function of each of the configuration elements. Therefore, a hardware configuration for use can be properly altered depending on a technology level of the time when the present disclosure is implemented.

It should be understood by those skilled in the art that various modifications, combinations, sub-combinations and alterations may occur depending on design requirements and other factors insofar as they are within the scope of the appended claims or the equivalents thereof.

Additionally, the following configurations are also within the technical scope of the present disclosure.

(1)

An information processing apparatus including:

a judgment unit for using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

(2)

The information processing apparatus according to (1),

wherein the judgment unit judges dilation/constriction of the blood vessel and/or increase/decrease of the blood flow by using reflectance of a first band corresponding to red and reflectance of a second band at a shorter wavelength side than the first band from among the reflectance of light obtained by irradiating with light belonging to a visible light band.

(3)

The information processing apparatus according to (1) or (2),

wherein the judgment unit judges a measurement portion of the living body to be in a condition of the blood vessel being dilated and the blood flow being increased when the reflectance of a wavelength belonging to the second band is a first threshold or less and a difference between the reflectance of a wavelength belonging to the first band and the reflectance of the wavelength belonging to the second band is a second threshold or more, and

wherein the judgment unit judges the measurement portion of the living body to be in a condition of the blood vessel being constricted and the blood flow being decreased when the reflectance of the wavelength belonging to the second band is a third threshold or more and a difference between the reflectance of the wavelength belonging to the first band and the reflectance of the wavelength belonging to the second band is a fourth threshold or less.

(4)

The information processing apparatus according to (3),

wherein the judgment unit changes setting values of the first, second, third, and fourth thresholds respectively depending on gender of the subject.

(5)

The information processing apparatus according to any of (1) to (4),

wherein the judgment unit uses, as the reflectance of the first band, at least one of reflectance at wavelength of 620 nm and reflectance at wavelength of 660 nm, and

wherein the judgment unit uses, as the reflectance of the second band, at least one of reflectance at wavelength of 500 nm, reflectance at wavelength of 540 nm, and reflectance at wavelength of 580 nm.

(6)

The information processing apparatus according to any of (2) to (5), further including:

a concentration calculator for calculating a concentration of a measurement target component at a measurement portion by using the measurement data in relation to the reflectance of light,

wherein the concentration calculator corrects the calculated concentration of the measurement target component in accordance with a judgment result of the judgment unit.

(7)

The information processing apparatus according to (6),

wherein the concentration calculator carries out the correction by dividing the calculated concentration of the measurement target component by a predetermined correction coefficient when it is judged by the judgment unit that the blood vessel is dilated and the blood flow is increased, and

wherein the concentration calculator carries out the correction by multiplying the calculated concentration of the measurement target component by a predetermined correction coefficient when it is judged by the judgment unit that the blood vessel is constricted and the blood flow is decreased.

(8)

The information processing apparatus according to any of (1) to (7), further including:

an application execution unit for executing an application that uses the judgment result of the judgment unit.

(9)

The information processing apparatus according to (8),

wherein the application execution unit judges whether or not there is stiffness of a body by using the judgment result of the judgment unit.

(10)

The information processing apparatus according to (8),

wherein the application execution unit specifies excitement of the subject by using the judgment result of the judgment unit, and reflects the specified excitement in a parameter of the application.

(11)

An information processing method including:

using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

(12)

A program for causing a computer to execute:

using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

(13)

An information processing system including:

a measurement unit for generating measurement data in relation to reflectance of light by irradiating a surface of a living body of a subject with light of a predetermined wavelength, and by detecting light reflected at the living body; and

an information processing apparatus including a judgment unit for judging a condition of a blood vessel and/or a condition of blood flow of the living body in accordance with a color phase corresponding to the reflectance of light by using the measurement data in relation to the reflectance of light generated by the measurement unit,

wherein the measurement unit includes
a light-receiving element at which light from a measurement target region is imaged, the surface of the living body being placed on the measurement target region; and
a plurality of light-emitting elements arranged on a periphery of the light-receiving element in a circular manner for irradiating the measurement target region with light of a predetermined wavelength, and

wherein the plurality of light-emitting elements is arranged to incline with respect to a normal line of the measurement target region so that a central line of irradiated light from each of the light-emitting elements passes through an approximate center of the measurement target region.

The present disclosure contains subject matter related to that disclosed in Japanese Priority Patent Application JP 2011-168191 filed in the Japan Patent Office on Aug. 1, 2011, the entire content of which is hereby incorporated by reference.

What is claimed is:

1. An information processing apparatus comprising:
a judgment unit for using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

2. The information processing apparatus according to claim 1,
wherein the judgment unit judges dilation/constriction of the blood vessel and/or increase/decrease of the blood flow by using reflectance of a first band corresponding to red and reflectance of a second band at a shorter wavelength side than the first band from among the reflectance of light obtained by irradiating with light belonging to a visible light band.

3. The information processing apparatus according to claim 2,
wherein the judgment unit judges a measurement portion of the living body to be in a condition of the blood vessel being dilated and the blood flow being increased when the reflectance of a wavelength belonging to the second band is a first threshold or less and a difference between the reflectance of a wavelength belonging to the first band and the reflectance of the wavelength belonging to the second band is a second threshold or more, and
wherein the judgment unit judges the measurement portion of the living body to be in a condition of the blood vessel being constricted and the blood flow being decreased when the reflectance of the wavelength belonging to the second band is a third threshold or more and a difference between the reflectance of the wavelength belonging to the first band and the reflectance of the wavelength belonging to the second band is a fourth threshold or less.

4. The information processing apparatus according to claim 3,
wherein the judgment unit changes setting values of the first, second, third, and fourth thresholds respectively depending on gender of the subject.

5. The information processing apparatus according to claim 3,
wherein the judgment unit uses, as the reflectance of the first band, at least one of reflectance at wavelength of 620 nm and reflectance at wavelength of 660 nm, and
wherein the judgment unit uses, as the reflectance of the second band, at least one of reflectance at wavelength of 500 nm, reflectance at wavelength of 540 nm, and reflectance at wavelength of 580 nm.

6. The information processing apparatus according to claim 2, further comprising:
a concentration calculator for calculating a concentration of a measurement target component at a measurement portion by using the measurement data in relation to the reflectance of light,
wherein the concentration calculator corrects the calculated concentration of the measurement target component in accordance with a judgment result of the judgment unit.

7. The information processing apparatus according to claim 6,
wherein the concentration calculator carries out the correction by dividing the calculated concentration of the measurement target component by a predetermined correction coefficient when it is judged by the judgment unit that the blood vessel is dilated and the blood flow is increased, and
wherein the concentration calculator carries out the correction by multiplying the calculated concentration of the measurement target component by a predetermined correction coefficient when it is judged by the judgment unit that the blood vessel is constricted and the blood flow is decreased.

8. The information processing apparatus according to claim 1, further comprising:
an application execution unit for executing an application that uses the judgment result of the judgment unit.

9. The information processing apparatus according to claim 8,
wherein the application execution unit judges whether or not there is stiffness of a body by using the judgment result of the judgment unit.

10. The information processing apparatus according to claim 8,
wherein the application execution unit specifies excitement of the subject by using the judgment result of the judgment unit, and reflects the specified excitement in a parameter of the application.

11. An information processing method comprising:
using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

12. A program for causing a computer to execute:
using measurement data in relation to reflectance of light obtained by irradiating a surface of a living body of a subject with light of a predetermined wavelength to judge a condition of a blood vessel and/or a condition of blood flow of the living body in accordance with a color phase corresponding to the reflectance of light.

13. An information processing system comprising:
a measurement unit for generating measurement data in relation to reflectance of light by irradiating a surface of a living body of a subject with light of a predetermined wavelength, and by detecting light reflected at the living body; and
an information processing apparatus including a judgment unit for judging a condition of a blood vessel and/or a condition of blood flow of the living body in accordance with a color phase corresponding to the reflectance of light by using the measurement data in relation to the reflectance of light generated by the measurement unit,
wherein the measurement unit includes
a light-receiving element at which light from a measurement target region is imaged, the surface of the living body being placed on the measurement target region; and
a plurality of light-emitting elements arranged on a periphery of the light-receiving element in a circular manner for irradiating the measurement target region with light of a predetermined wavelength, and
wherein the plurality of light-emitting elements is arranged to incline with respect to a normal line of the measurement target region so that a central line of irradiated light from each of the light-emitting elements passes through an approximate center of the measurement target region.
